(19) United States
(12) Patent Application Publication
(10) Pub. No.: US 2025/0206703 A1
(43) Pub. Date: Jun. 26, 2025

(54) PROCESS FOR PREPARING ((1S,2S)-2-(5-METHYLPYRIDIN-2-YL)CYCLOPROPYL)METHANOL

(71) Applicant: Merck Sharp & Dohme LLC, Rahway, NJ (US)

(72) Inventors: Juan D. Arredondo, Brooklyn, NY (US); Yang Cao, Scotch Plains, NJ (US); John Y. L. Chung, Edison, NJ (US); Hongmei Li, Potomac, MD (US)

(73) Assignee: Merck Sharp & Dohme LLC, Rahway, NJ (US)

(21) Appl. No.: 18/851,288

(22) PCT Filed: Mar. 27, 2023
(86) PCT No.: PCT/US2023/016396

§ 371 Date: Sep. 26, 2024

Related U.S. Application Data
(60) Provisional application No. 63/326,327, filed on Apr. 1, 2022.

(51) Int. Cl. C07D 213/30; C07D 213/50; C07D 213/55; C12N 9/04

(52) U.S. Cl. CPC C07D 213/30; C07D 213/50; C07D 213/55; C12N 9/0006

(57) ABSTRACT

Described is an efficient, scalable synthesis of compounds such as ((1S,2S)-2-(5-methylpyridin-2-yl)cyclo-propyl)m ethanol, which contains a disubstituted cyclopropane with two stereogenic centers and represents a significant challenging synthetic target.

### BACKGROUND OF THE INVENTION

The present invention relates to an efficient scalable synthesis of ((1S,2S)-2-(5-methylpyridin-2-yl)cyclo-propyl)methanol (Compound 5) and structurally related compounds. Compound 5 contains a disubstituted cyclopropane with two stereogenic centers and represents a significant challenging synthetic target. WO2013/028590 discloses a 5-step synthetic route to Compound 5. Although this route is able to generate kilogram quantities of Compound 5, it is not suitable for large scale manufacturing due to: (1) potential safety/explosion hazard of ethyl diazoacetate at elevated reaction temperatures (135° C.); (2) lack of diastereo- and enantio-control (de and ee) for the cyclopropanation; (3) low efficiency in silica and SFC chromatographies to remove undesired stereoisomers; and (4) poor overall yield (15%). Therefore, a more efficient and safe synthesis that avoids or improves these issues is desired.

### SUMMARY OF THE INVENTION

Provided is a safe and efficient process for making compounds such as Compound 5. In one embodiment, disclosed is a process for making compounds such as Compound 5 that reduces or eliminates the explosion hazard associated with use of ethyl diazoacetate. Another embodiment is a process for making compounds such as Compound 5 wherein control of diastereomeric excess (de) and enantioselectivity excess (ee) is achieved. A subembodiment of this aspect is a process wherein the chiral purity of compounds such as Compound 5 can be achieved in >99.5% de and >99.5% ee. Another subembodiment of this aspect is a process that results in compounds such as Compound 5 with less than 0.25% cis isomers detected after crystallization. Another embodiment is a process for making compounds such as Compound 5 that requires no chromatographic purification. Another embodiment is a process for making compounds such as Compound 5 with a yield of at least 50%. Another embodiment is a process for making compounds such as Compound 5 in kilogram quantities with a yield of at least 50% and purity of 99.9% LCAP (liquid chromatography area percent]) and 99.9% ee in four steps. This and other aspects of the present disclosure are realized upon review of the entire specification.

### DETAILED DESCRIPTION OF THE INVENTION

The presence of two stereogenic centers on compounds such as the disubstituted cyclopropane ((1S,2S)-2-(5-methylpyridin-2-yl)cyclo-propyl)methanol (Compound 5) makes it a significantly challenging synthetic target. Disclosed herein is a process for making a compound of formula 5′

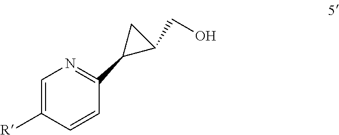

comprising the steps of:
1) reacting a compound of formula (7′),

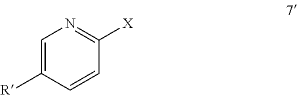

with an organometallic species and an alkyl amide represented by 7a′,

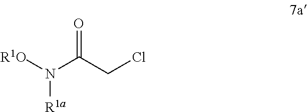

to produce the compound of structural formula 6′,

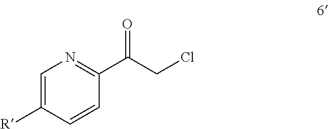

wherein X is selected from halogen, and R′ is selected from
   (1) hydrogen,
   (2) halogen,
   (3) C₁₋₁₀ alkyl,
   (4) C₀₋₆ alkylOR\hat{ },
   (5) C₀₋₆ alkylSR\hat{ },
   (6) C₀₋₃ haloalkyl, and
   (7) C₆₋₁₀ aryl, and
R\hat{ }, R¹ and R^{1a} independently are C₁₋₆ alkyl,
2) reacting the compound of formula 6′ with a reducing enzyme at a pH of about 5 to about 9 and temperature of about 10° C. to about 50° C. to produce a compound of structural formula 1′

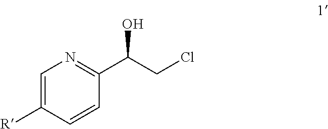

3) adding a non-nucleophilic base and phosphonate agent to the compound of formula 1′ to produce a compound of formula 4′

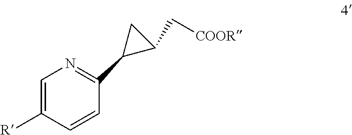

wherein R″ is selected from
   (1) hydrogen,
   (2) halogen,
   (3) C₁₋₁₀ alkyl,
   (4) C₀₋₆ alkylOR\hat{ },
   (5) C₀₋₆ alkylSR\hat{ },
   (6) C₀₋₃ haloalkyl, and
   (7) C₆₋₁₀ aryl,
4) reducing the compound of formula 4′ using a reducing agent to produce the compound of formula 5′ and isolating the compound of structural formula 5′.

Another embodiment of this process is realized when X is selected from bromine, chlorine, fluorine, and iodine. An aspect of this embodiment is realized when X is bromine. Another aspect of this embodiment is realized when X is fluorine. Another aspect of this embodiment is realized when X is chlorine. Yet another aspect of this embodiment is realized when X is iodine.

Another embodiment of this invention is realized when R′ is selected from methyl, ethyl, propyl, butyl, pentyl, hexyl, —OR\hat{ }, SRA, and C₆₋₁₀ aryl. An aspect of this embodiment is realized when R′ is selected from methyl, ethyl, and propyl. Another aspect of this embodiment is realized when R′ is methyl. Still another aspect of this embodiment is realized when R′ is ethyl. Yet another aspect of this embodiment is realized when R′ is propyl. Another aspect of this embodiment is realized when R′ is butyl. Another aspect of this embodiment is realized when R′ is pentyl. Another aspect of this embodiment is realized when R′ is hexyl. Another aspect of this embodiment is realized when R′ is OR\hat{ }. Another aspect of this embodiment is realized when R′ is SRA. Another aspect of this embodiment is realized when R′ is C₆₋₁₀ aryl.

Another embodiment of this process is realized when the organometallic species is selected from, alkyl magnesium halide, allyl-magnesium halide, vinyl-magnesium halide, aryl-magnesium halide, magnesium, nHexLi/ZnCl₂/CuCl, iPrMgCl—LiCl, and alkyl lithium (e.g., n-butyl lithium, sec-butyl lithium, and tert-butyl lithium, hexyl-lithium) or derivative thereof. A subembodiment of this aspect of the process is realized when the organometallic species is selected from iPrMgCl, iPrMgCl—LiCl, n-hexyl-lithium (nHexLi), magnesium (Mg), and nHexLi/ZnCl₂/CuCl. Another subembodiment of this aspect of the disclosure is realized when the organometallic species is selected from iPrMgCl, iPrMgCl—LiCl, and nHexLi. Another subembodiment of this aspect is realized when the organometallic species is iPrMgCl. Another subembodiment of this aspect of the process is realized when the organometallic species is iPrMgCl—LiCl. Still another subembodiment of this aspect of the process is realized when the organometallic species is nHexLi.

Another embodiment of this process is realized when the temperature of Step 1 is maintained from about room temperature (rt) to about −80° C., from about 0° C. to about −50° C., or from about −20° C. to about −35° C. A subembodiment of this process is realized when the temperature in Step 1 is maintained at less than −10° C. during addition of the organometallic species.

Use of enzymes in the synthesis of chiral alcohols are known (see Ling He, Dongzhi Wei, et al., https://doi.org/10.1021/acs.oprd.1c00189; and Ambarish Singh et al., Org. Proc. Res. & Dev. 2002, 6, 618-620). Reducing enzymes useful for this process can be obtained from commercially available sources, for example Codexis. An embodiment of this process is realized when the reducing enzyme is selected from NADH, or KRED P3D1, P3D1, P1H8, P1H1, P3C₃, CDX004, CDX005, CDX025, or CDX026, in combination with co-enzyme NAD(P). A subembodiment of this aspect of the process is realized when the reducing enzyme is NADH. A subembodiment of this aspect of the process is realized when the reducing enzyme is KRED P3D1. A subembodiment of this aspect of the process is realized when the reducing enzyme is P3D1. A subembodiment of this aspect of the process is realized when the reducing enzyme is P1H8. A subembodiment of this aspect of the process is realized when the reducing enzyme is P1H1. A subembodiment of this aspect of the process is realized when the reducing enzyme is P3C₃. A subembodiment of this aspect of the process is realized when the reducing enzyme is CDX004. A subembodiment of this aspect of the process is realized when the reducing enzyme is CDX005. A subembodiment of this aspect of the process is realized when the reducing enzyme is CDX025. A subembodiment of this aspect of the process is realized when the reducing enzyme is CDX026. See Discloses a biocatalytic synthesis of (R)-2-Chloro-1-(3,4-difluorophenyl)ethanol by the short-chain dehydrogenase PpKR8 from Paraburkholderia phymatum. %

Another embodiment of this process is realized when Step 2 is conducted at a temperature of about 20° C. to about 40° C., preferably about 30° C.

Another embodiment of this process is realized when Step 2 is conducted at a pH of about 6.0 to about 7.0.

Another embodiment of this process is realized when the non-nucleophilic base is selected from sodium tert-butoxide (NaOtBu), potassium tert-butoxide (KOtBu), sodium bis(trimethylsilyl)amide (NaHMDS), potassium bis(trimethylsilyl)amide (KHMDS), lithium diisopropylamide (LDA), 1,8-diazabicyclo[5.4.0]undec-7-ene (DBU), tetramethylethylene diamine and lithium tetramethylpiperidide (LiTMP). A subembodiment of this process is realized when the non-nucleophilic base is sodium tert-butoxide (NaOtBu). A subembodiment of this process is realized when the non-nucleophilic base is potassium tert-butoxide (KOtBu). A subembodiment of this process is realized when the non-nucleophilic base is sodium bis(trimethylsilyl)amide (NaHMDS). A subembodiment of this process is realized when the non-nucleophilic base is potassium bis(trimethylsilyl)amide (KHMDS). A subembodiment of this process is realized when the non-nucleophilic base is lithium diisopropylamide (LDA). A subembodiment of this process is realized when the non-nucleophilic base is 1,8-diazabicyclo[5.4.0]undec-7-ene (DBU). A subembodiment of this process is realized when the non-nucleophilic base is tetramethylethylene diamine. A subembodiment of this process is realized when the non-nucleophilic base is lithium tetramethylpiperidide (LiTMP).

Another embodiment of this process is realized when the phosphonate agent is selected from trimethyl phosphonoacetate, triethyl phosphonoacetate, tributyl phosphonoacetate, triphenyl phosphonoacetate, propyl dibutylphosphonate, tertbutyl diethylphosphonoacetate, and pentyl dibutylphosphonoacetate. A subembodiment of this process is realized when the phosphonate agent is trimethyl phosphonoacetate. A subembodiment of this process is realized when the phosphonate agent is triethyl phosphonoacetate. A subembodiment of this process is realized when the phosphonate agent is tributyl phosphonoacetate. A subembodiment of this process is realized when the phosphonate agent is triphenyl phosphonoacetate. A subembodiment of this process is realized when the phosphonate agent is propyl dibutylphosphonate. A subembodiment of this process is realized when the phosphonate agent is pentyl dibutylphosphonoacetate. A subembodiment of this process is realized when the phosphonate agent is tertbutyl diethylphosphonoacetate.

Another embodiment of this process is realized when Step 3 is conducted in the presence of an anhydrous solvent. A subembodiment of this aspect of the process is realized when the anhydrous solvent is selected from THF, 2-MeTHF, ether, hexane, MTBE, and DMPU, or mixtures thereof. A subembodiment of this aspect of the process is realized when the anhydrous solvent is THF. A subembodiment of this aspect of the process is realized when the anhydrous solvent is 2-MeTHF. A subembodiment of this aspect of the process is realized when the anhydrous solvent is ether. A subembodiment of this aspect of the process is realized when the anhydrous solvent is hexane. A subembodiment of this aspect of the process is realized when the anhydrous solvent is MTBE. A subembodiment of this aspect of the process is realized when the anhydrous solvent is DMPU.

Another embodiment of this process is realized when the ratio of formula 1′ to non-nucleophilic base and phosphonate agent is about 1:1.7:3.2, 1:1.7:2.0, 1:1.8:20, 1:1.8:2.2, 1:1.9:2.0, 1:2.0:2.0, 1:2.0:2.2, or 1:2.0:3.0 equivalents, respectively. A subembodiment of this aspect of the invention is realized when the ratio of formula 1′ to non-nucleophilic base and phophonate agent is about 1:2.0:2.2 equivalents, respectively.

Step 3 involves in-situ production of a cyclo-propoxy building block (epoxide) which is then transformed to a compound of formula 4′. Xu et al., Org. Lett. 2017, 19, 5880-5883. Another embodiment of this process is realized in Step 3 when the addition of the non-nucleophilic base and phosphonate agent to the compound of formula 1′ in the presence of an anhydrous solvent initially produces an epoxide which is then transformed to a compound of formula 4′. A subembodiment of this aspect of the process is realized when the epoxide is produced in-situ without isolation.

Another embodiment of this process is realized when the reducing agent is selected from LiAlH₄, NaBH₄, BH₃, and dihydrogen (H₂). A subembodiment of this process is realized when the reducing agent is LiAlH₄. A subembodiment of this process is realized when the reducing agent is NaBH₄. A subembodiment of this process is realized when the reducing agent is selected from BH₃. A subembodiment of this process is realized when the reducing agent is dihydrogen in the presence of a hydrogenation catalyst.

Another embodiment of this process is realized when the chiral purity of compound of formula 5′ is improved in Step 4 by reaction with toluene, heptane and/or mixture thereof.

As used herein, “alkyl” refers to both branched- and straight-chain saturated aliphatic hydrocarbon groups having the specified number of carbon atoms in a specified range. For example the term “C₁₋₁₀ alkyl” means linear or branched chain alkyl groups, including all possible isomers, having 1, 2, 3, 4, 5, 7, 8, 9 or 10 carbon atoms, and includes each of the decyl, nonyl, octyl, heptyl, hexyl and pentyl isomers as well as n-, iso-, sec- and tert-butyl (butyl, s-butyl, i-butyl, t-butyl; Bu=butyl, collectively “—C₄alkyl”), n- and iso-propyl (propyl, i-propyl, Pr=propyl, collectively “—C₃alkyl”), ethyl (Et) and methyl (Me). “C₁₋₄alkyl” has 1, 2, 3 or 4 carbon atoms, and includes each of n-, iso-, sec- and tert-butyl, n- and i-propyl, ethyl and methyl.

As used herein, “aryl” refers to (i) phenyl, (ii) 9- or 10-membered bicyclic, fused carbocylic ring systems in which at least one ring is aromatic, and (iii) 11- to 14-membered tricyclic, fused carbocyclic ring systems in which at least one ring is aromatic. Suitable aryls include, for example, substituted and unsubstituted phenyl and substituted and unsubstituted naphthyl. An aryl of particular interest is unsubstituted or substituted phenyl.

### EXAMPLES

### General Description

Solvents, reagents, and intermediates that are commercially available were used as received. Reagents and intermediates that are not commercially available were prepared in the manner as described herein. ¹H NMR spectra are reported as ppm downfield from Me₄Si with number of protons, multiplicities, and coupling constants in Hertz indicated parenthetically. Examples of organic solvents useful for this process are THF, 2-MeTHF, MTBE, ethanol, propanol, isopropanol, acetonitrile, acetone, heptane, hexane, toluene, methanol, or mixtures thereof.

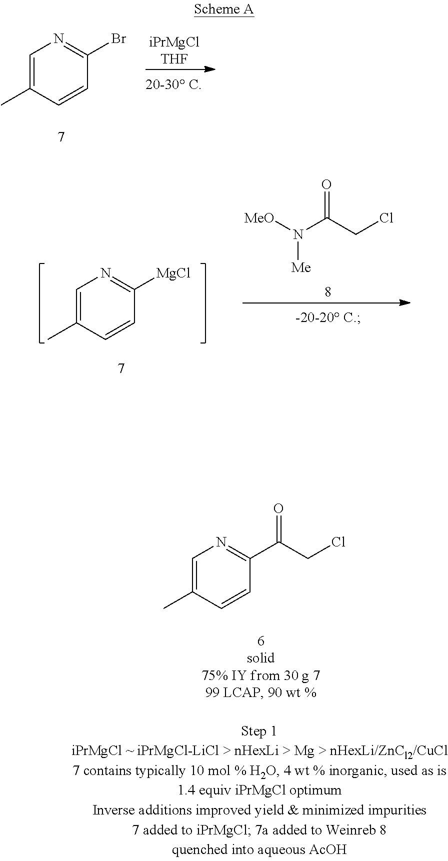

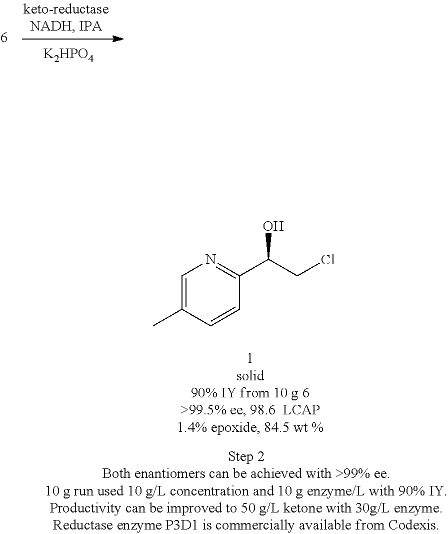

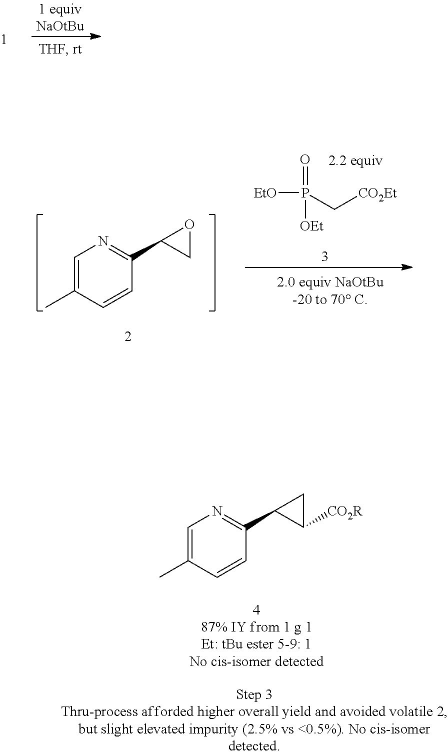

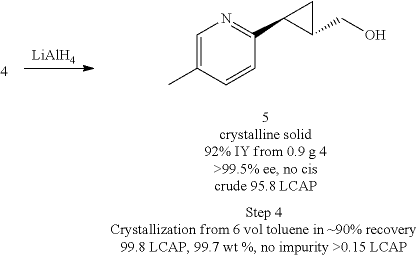

wherein IY=isolated yield

As illustrated in Scheme A, the instant process relates to a 4-step chemoenzymatic route for making efficient and scalable compounds such as compound 5, ((1S,2S)-2-(5-methylpyridin-2-yl)cyclo-propyl)methanol. Step 1 is conversion of 7 to chloromethylketone 6 via Grignard 7a. Step 2 is an enzymatic reduction of the ketone to chlorohydrine 1 which can be isolated in about 90% yield with near perfect enantioselectivity. Step 3 is conversion of chlorohydrine 1 to cyclopropane 4 with a phosphonate in the presence of a non-nucleophilic base via an epoxide, which can be done in a one-pot through process. Finally, Step 4 is reduction of 4 to 5, which can be isolated in 92% yield with >99.5% ee with no cis isomers detected after crystallization. Features of this 4-step route to 5 included complete control of diastereo-/enantioselectivity, no chromatographic purification and 3.6-fold overall yield improvement (54% vs 15% (as disclosed in WO2013/028590)).

Another embodiment of this process is realized when about 90% yield is achieved with the enzymatic reduction of the ketone in Step 2 to produce formula 1′. Another aspect of this embodiment of the process is realized when >99.5% enantioselectivity is achieved with the enzymatic reduction of the ketone in the second step to produce formula 1. Still another embodiment of this process is realized when about 92% yield with >99.5% ee is achieved in Step 4 reduction of ester 4 to 5. Another aspect of this embodiment of the process is realized when less than 0.25% cis isomers are detected after crystallization.

### Example 1: Synthesis of 2-Chloro-1-(5-methylpyridin-2-yl)ethanone

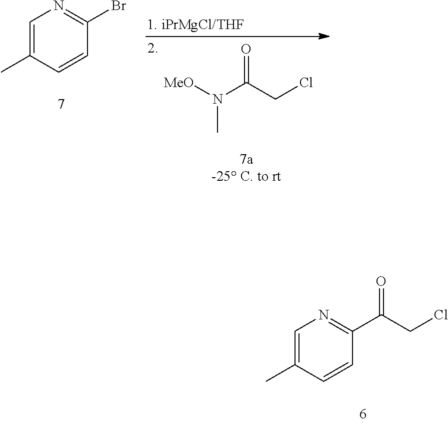

| Materials | MW | Amt. | | mmol | Equiv |
|---|---|---|---|---|---|
| 2-Bromo-5-methylpyridine (7) | 172.02 | 30 | g | 174 | 1.00 |
| Isopropyl magnesium chloride | 102.85 | 140 | mL | 281.4 | 1.61 |
| (2M) | | | | | |
| 2-Chloro-N-methoxy-N- | 137.57 | 34.6 | g | 239.2 | 1.37 |
| methylacetamide (7a) | | | | | |

A three neck flask (1 L) equipped with mechanical stirrer, thermocouple, and nitrogen sweep was charged with 2M isopropyl magnesium chloride in THF (100 mL, 1.15 eq), followed by the addition of 2-bromo-5-methylpyridine (30 g) in 120 mL anhydrous THF (4 vol, KF as 36 ppm) over 20 min at room temperature (rt). The resulting solution was stirred at room temperature. HPLC/¹HNMR (CD30D/THF-d₈) indicated ˜35% 7 left, with no more isopropyl magnesium chloride after overnight. Additional iPrMgCl (40 mL, 0.46 eq) was added at rt, and the solution was stirred until HPLC/¹HNMR (CD30D) confirmed >99% conversion (˜5.5 h).

In another three neck flask (1 L) equipped with mechanical stirrer, thermocouple, cooling bath, and nitrogen sweep was charged with 2-Chloro-N-methoxy-N-methylacetamide 7a (32.8 g, 227 mmol, 1.3 eq), followed by the addition of anhydrous THF (150 mL, 5 vol) at rt. The resulting solution was then cooled down to −30° C.

The Grignard reagent was cooled down to −30° C., and cannulated into the cold 7a solution over 30 min, followed by 20 mL THF rinse (Tᵢ was controlled <−25° C. during the addition). The resulting solution was allowed to warm to rt gradually and stirred at rt. HPLC indicated ˜6.8% 3-picoline after overnight stirring, another 0.07 eq of 7a, 2-Chloro-N-methoxy-N-methylacetamide (1.768 g, 12.21 mmol), was added at rt. After 2 hours (h), HPLC showed 6.2% 3-picoline, the reaction was deemed as complete.

In another flask (2 L) equipped with mechanical stirrer, thermocouple, and cooling bath was charged with water (400 mL) and acetic acid (3 eq to 2-bromo-5-methylpyridine (7), 31.2 g), cooled down to 0° C. The reaction mixture was cannulated into the acetic acid solution over 20 min, Tᵢ was controlled <0° C. After quench, the biphasic solution was allowed to warm up to 10° C. (pH˜5.35). The pH was adjusted pH to 7 by the addition of solid NaHCO₃. 500 mL MTBE was added, the organic layer was washed with water (200 mL×2), brine (200 mL), dried over Na₂SO₄, filtered, and concentrated to give 32.83 g crude solid 6. ¹HNMR indicated 72.6 wt %, 23.83 g, calculated as 80.5% AY (assay yield). The crude solid was triturated with Heptane (50 mL), filtered, then slurry washed with Heptane (20 mL) to give a solid, 27.83 g, HPLC assay indicated 79.2 wt %, 22.04 g, calculated as 74.5% AY.

¹H NMR (500 MHz, CDCl₃) δ: 8.47 (s, 1H), 7.99-8.01 (d, 1H), 7.65-7.67 (d, 1H), 5.08 (s, 2H), 2.42 (s, 3H).

LC method: Column: Supelco Ascentis Express C18 10 cm×4.6 mm×2.7 um; Flow rate 1.0 mL/min; Detector 210 nm; A=5 mM (NH₄)₂HPO₄: MeOH (75:25); B=ACN: MeOH (75:25); Gradient: 20% B for 1 min, then to 95% B over 5 min, hold for 2 min at 95%; Post-time: 2 minutes; Injection: 5 μL; Column Temperature: 25° C.

| Compound | retention time |
|---|---|
| 2-Bromo-5-methylpyridine | 3.81 min |
| 3-Picoline | 1.92 min |
| Chloro-ketone | 4.32 min |
| Chlorohydrin | 2.26 min |

### Example 2: Synthesis of (R)-2-chloro-1-(5-methylpyridin-2-yl)ethan-1-ol

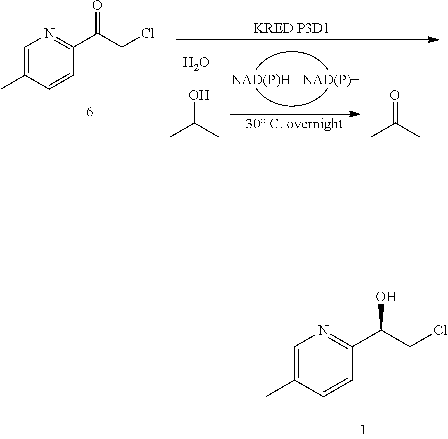

| Materials | MW | Amt. | mmol | Equiv |
|---|---|---|---|---|
| Chloroketone 6 | 169.61 | 10 g (90.4 wt %) | 53.3 | 1.00 |
| P3D1 | | 10 g | | 100 wt % |
| NADP+ | 744.41 | 1.0 g | | 2.5% |

In a 2 L three neck flask equipped with overhead stirring, thermocouple, and heating mantle, 1.0 g of KRED P3D1 and 1.0 g of NADP were dissolved in 1 L of pH 7.0 K₂HPO₄ buffer (0.1M). The stock solution of chloroketone (10 g, 90.4 wt %, 53.3 mmol) in 300 mL 2-Propanol was charged, and the reaction mixture was aged at 30° C. overnight. One mL of the reaction mixture was diluted with 0.5 mL MTBE, vortexed and centrifuged. Ten μL of the organic layer was diluted to 1 mL by 50:50 water/ACN for HPLC assay, the HPLC indicated full conversion. SFC for the organic layer indicated >99% ee. The reaction mixture was diluted with 250 mL of MTBE and layers were cut. The aqueous layer was back-extracted with 250 mL of 2:1 MTBE:IPA solution. The combined organic layers were washed with 100 mL of brine, dried over Na₂SO₄, filtered and concentrated to dryness to give 9.77 g oil which solidified slowly to crystalline solid. HPLC indicated 98.6 LCAP, 84.5 wt %, 90.2% IY (isolated yield). The material was used in next step without further purification.

¹H NMR (500 MHz, CDCl₃) δ: 8.39 (s, 1H), 7.52-7.54 (d, 1H), 7.27-7.29 (m, 1H), 4.91-4.93 (m, 1H), 4.27 (s, 1H, —OH), 3.74-3.84 (m, 2H), 2.36 (s, 3H).

¹³C NMR (126 MHz, CDCl₃) δ: 154.3, 149.9, 137.3, 132.7, 119.3, 52.8, 50.3, 18.2.

HPLC method: Column: Supelco Ascentis Express C18 10 cm×4.6 mm×2.7 um; Flow rate 1.0 mL/min

Detector 210 nm; A=5 mM (NH₄)₂HPO₄: MeOH (75:25); B=ACN: MeOH (75:25); Gradient: 20% B for 1 min, then to 95% B over 5 min, hold for 2 min at 95%; Post-time: 2 minutes;

Injection: 5 μL; Column Temperature: 25° C.

| Compound | retention time |
|---|---|
| Chloroketone | 4.32 min |
| Chlorohydrin | 2.26 min |

Chiral supercritical fluid chromatography (SFC) method (Aurora): Chlorohydrin, Column: AD-H, 250×4.6 mm, 5 um; Condition: isocratic 10% EtOH with 25 mM IBA, 200 bar; Flow rate: 3 mL/min; Detector: 210 nm. The absolute configuration of the chlorohydrin was determined as (R) by X-ray crystallography (mixed solvents Toluene: Heptane=1:1 were used to grow the crystals for X-ray study).

| Compound | retention time |
|---|---|
| (R)-Chlorohydrin | 4.76 min |
| (S)-Chlorohydrin | 4.40 min |

### Example 3: Synthesis of (S,S)-ethyl 2-(5-methylpyridin-2-yl)cyclopropanecarboxylate

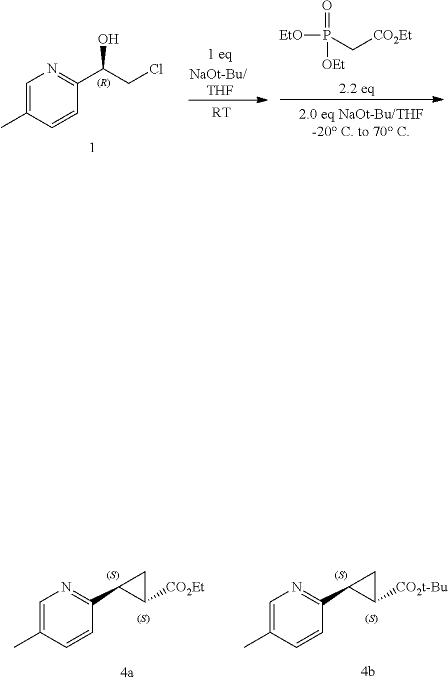

The crude chlorohydrin obtained from enzymatic reduction (KRED P3D1) analyzed as 98.6 LCAP, with 1.4% epoxide, KF as 946 ppm, was used directly without further purification. A three neck flask (100 mL) equipped with magnetic stirrer, thermocouple, and nitrogen sweep was charged with chlorohydrin (6.67 g, 5.633 g pure, 32.8 mmol), followed by 40 mL anhydrous THF (40 mL, inhibitor free, KF 20 ppm). NaOtBu (16.41 mL, 32.8 mmol) was added dropwise over 6 minutes (min) at rt (10° C. exotherm), the orange solution turned into light brown suspension. After 20 min, 5 NL of the organic layer was diluted to 1 mL by 50% water/acetonitrile for HPLC assay, the HPLC showed 92.6% conversion, indicating the material is ready for cyclopropanation.

In another 250 mL 3-neck flask with mechanical stirrer, condenser, and N₂ inlet was charged with triethyl phosphonoacetate (14.53 mL, 72.2 mmol), followed by the addition of anhydrous THF (40 mL) at rt, then cooled down to −20° C. NaOtBu (32.8 mL, 65.6 mmol) was added dropwise over 5 min, with Tᵢₙₜₑᵣₙₐₗ controlled between −20° C. to −26° C. After addition, the solution was allowed to warm up to 0° C. over 30 min, then cooled back to −10° C. The epoxide mixture was added into the anion over 30 min, with Tᵢ controlled between −10° C. to −15° C. After addition, the solution was allowed to warm up to 0° C. over 30 min, then heated on oil bath to 62° C. for 1 h, followed by 70° C. reflux overnight (total heating 17 hours) to achieve complete conversion, with ˜2.6% eliminated by product (ethyl (E)-3-(5-methylpyridin-2-yl)but-2-enoate which was confirmed by NMR), and 5% t-Bu ester 4b (18.4:1 Et:t-Bu ester).

The reaction mixture was cooled on ice bath to 0-2° C., diluted with 100 mL MTBE, and 100 mL water (Tᵢ<10° C.), stirred at 5° C. for 5 min. The layers were separated. The aqueous layer (pH 12-13) was extracted with 100 mL MTBE. The combined organic phase was washed with water, brine, dried over Na₂SO₄, filtered, and concentrated to give an oil, 10.11 g.

The crude oil was cooled on ice bath, taken into 50 mL 2N HCl, extracted with MTBE (50 mL×2), the aqueous layer was cooled on ice batch, adjusted pH to ˜12 using 5N NaOH (25 mL), extracted with MTBE (100 mL, then 50 mL×2). The organic layer was washed with water, brine, dried over Na₂SO₄, filtered, and concentrated to give 7.66 g oil 4a, 82.9 wt %, calculated as 94.2% assay yield. The crude oil was flushed with N₂, and kept in the refrigerator.

### Example 4: Synthesis of (S,S)-2-(5-methylpyridin-2-yl)cyclopropyl methanol

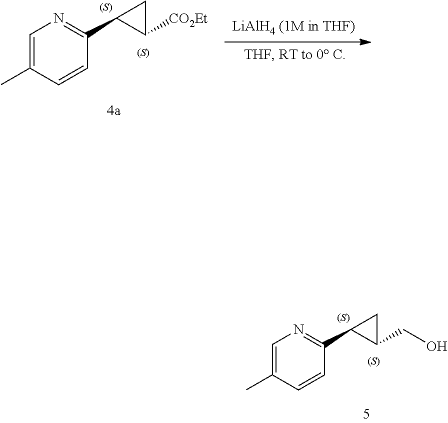

In a 500 mL 3-neck flask, equipped with mechanical stirrer, temperature probe, and N₂ inlet was charged (1S,2S)-ethyl 2-(5-methylpyridin-2-yl)cyclopropanecarboxylate 4a (7.46 g, 30.1 mmol), followed by the addition of 70 mL THF. The solution was cooled on ice bath. LiAlH₄ (1 M) in THF (32.5 mL, 32.5 mmol) was added over 30 min, Tᵢ was controlled to <5° C. After addition, stirred at rt overnight. After overnight, HPLC confirmed complete conversion. The solution was cooled down on ice bath, 3.5 g Solka Floc powdered cellulose filter aid (50 wt %) was added, then 1.3 mL water was added slowly with Tᵢ<10° C., followed by 5 N NaOH (2.6 mL), and 3.9 mL water. The slurry was filtered, and the cake was slurry washed with ethyl acetate (20 mL×3). The combined filtrate was concentrated to dryness, and solvent switched to toluene, concentrated to a solid 5, 5.30 g. HPLC assay indicated 92 wt %, calculated as 4.88 g pure, 99.2% assay yield.

### Example 5: Recrystallization of (S,S)-2-(5-methylpyridin-2-yl)cyclopropyl methanol

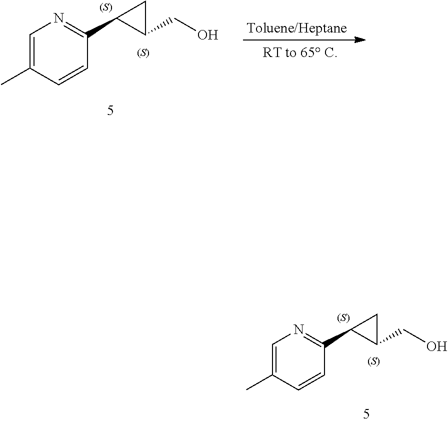

In a 100 mL jacket 3-neck flask, equipped with mechanical stirrer, temperature probe, and N₂ inlet was charged crude (1S,2S)-2-(5-methylpyridin-2-yl)cyclopropyl methanol 5 (assayed as 4.474 g, 27.4 mmol), followed by the addition of 13.42 mL toluene (3 vol). The slurry was heated to 65° C. over 1 hour to dissolve the solid, followed by the addition of heptane (4.47 mL, 1 vol) at 65° C. The clear solution was gradually cooled down and stirred at rt overnight. At 23° C., 10 μL supernatant was diluted to 1 mL (50% water/ACN (acetonitrile), 0.1% HCOOH) for HPLC analysis, which indicated 11.8 mg/mL, a loss of 212 mg, with ˜24 mg cis, 67 mg eliminated byproduct, 134 mg unknown impurity (RT 4.53 min) (total volume as 18 mL) (all the impurities were rejected in the supernatant). Another 0.5 vol of heptane (2.3 mL) was added over 10 min, stirred at rt for 30 min, supernatant checked by HPLC indicated 9 mg/mL, loss as 182 mg, with ˜28.5 mg cis, 67 mg byproduct, 139 mg unknown (Total volume as 20.3 mL). Another 0.5 vol of heptane (2.3 mL, total 2 vol) was added over 10 min, stirred at rt for 30 min, supernatant checked by HPLC indicated 7.5 mg/mL, loss as 170 mg, with ˜26.2 mg cis, 64 mg byproduct, 133 mg unknown (Total volume as 22.6 mL).

The crystals were filtered. The cake was washed with 2 vol of 1:1 toluene/heptane, dried to give 4.277 g solid 5, HPLC assay indicated >99.5 LCAP, 98.5 wt %, 94.3% isolated yield. Chiral SFC indicated >99.8% e.e, no undesired isomer was detected.

1. A process for making a compound of formula 5′ 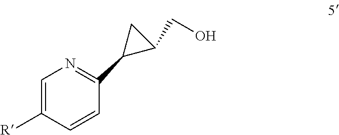
comprising the steps of:
1) reacting a compound of formula (7′), 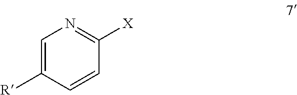
with an organometallic species and an alkyl amide represented by 7a′, 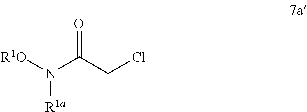
to produce the compound of structural formula 6′, 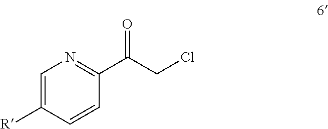
wherein X is selected from halogen, alkyl sulfonate, or aryl sulfonate, and R′ is selected from
(1) hydrogen,
(2) halogen,
(3) C₁₋₁₀ alkyl,
(4) C₀₋₆ alkylOR\hat{ },
(5) C₀₋₆ alkylSR\hat{ },
(6) C₀₋₃ haloalkyl,
(7) C₆₋₁₀ aryl, and
R\hat{ }, R¹ and R^{1a} independently are C₁₋₆ alkyl,
2) reacting the compound of formula 6′ with a reducing enzyme at a pH of about 5 to about 9 and temperature of about 10° C. to about 50° C. to produce a compound of structural formula 1′ 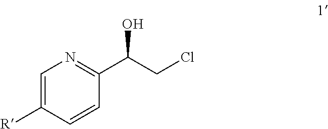
3) adding a non-nucleophilic base and phosphonate agent to the compound of formula 1′ to produce a compound of formula 4′ 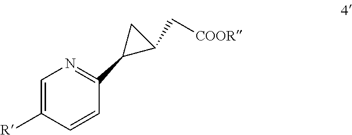
wherein R″ is selected from
(1) hydrogen,
(2) halogen,
(3) C₁₋₁₀ alkyl,
(4) C₀₋₆ alkylOR\hat{ },
(5) C₀₋₆ alkylSR\hat{ },
(6) C₀₋₃ haloalkyl, and
(7) C₆₋₁₀ aryl,
4) reducing the compound of formula 4′ using a reducing agent to produce the compound of formula 5′ and isolating the compound of structural formula 5′.

2. The process according claim 1 wherein X is selected from bromine, chlorine, fluorine, and iodine and R′ is selected from methyl, ethyl, propyl, butyl, pentyl, hexyl, —OR\hat{ }, SR\hat{ }, and C₆₋₁₀ aryl.

3. The process according to claim 1 wherein R′ is methyl.

4. The process according to claim 1 wherein the organometallic species is selected from, alkyl magnesium halide, allyl-magnesium halide, vinyl-magnesium halide aryl-magnesium haliden-butyl lithium, sec-butyl lithium, tert-butyl lithium, hexyl lithium.

5. The process according to claim 1 wherein the organometallic species is selected from iPrMgCl, iPrMgCl—LiCl, n-hexyl-lithium, magnesium, and nHexLi/ZnCl₂/CuCl.

6. The process according to claim 1 wherein the temperature in Step 1 is maintained from about 0° C. to about −50° C.

7. The process according to claim 6 wherein the temperature in Step 1 is maintained at less than −20° C.

8. The process according to claim 1 wherein the reducing enzyme is independently selected from NADH, KRED P3D1, P3D1, P1H8, P1H1, P3C₃, CDX004, CDX005, CDX025, and CDX026.

9. The process according to claim 1 wherein Step 2 is conducted at a temperature of about 20° C. to about 40° C.

10. The process according to claim 1 wherein Step 2 is conducted at a pH of about 6.0 to about 7.0.

11. The process according to claim 1 wherein about 90% yield is achieved with the enzymatic reduction of 6′ in Step 2 to produce formula 1′.

12. The process according to claim 1 wherein >99.5% enantioselectivity is achieved with the enzymatic reduction of the 6′ in Step to produce formula 1.

13. The process according to claim 1 wherein the non-nucleophilic base is selected from sodium tert-butoxide potassium tert-butoxide, sodium bis(trimethylsilyl)amide, potassium bis(trimethylsilyl)amide, lithium diisopropylamide, 1,8-Diazabicyclo[5.4.0]undec-7-ene, tetramethylethylene diamine and lithium tetramethylpiperidide.

14. The process according to claim 1 wherein the phosphonate agent is selected from trimethyl phosphonoacetate, triethyl phosphonoacetate, tributyl phosphonoacetate, triphenyl phosphonoacetate, propyl dibutylphosphonate, tertbutyl diethylphosphonoacetate, and pentyl dibutylphosphonoacetate.

15. The process according to claim 1 wherein Step 3 is conducted in the presence of anhydrous solvent selected from THF, 2-MeTHF, ether, hexane, MTBE, and DMPU, or mixtures thereof.

16. The process according to claim 1 wherein the ratio of formula 1′ to non-nucleophilic base and phosphonate agent is selected from: about 1:1.7:3.2, about 1:1.7:2.0, about 1:1.8:20, about 1:1.8:2.2, about 1:1.9:2.0, about 1:2.0:2.0, about 1:2.0:2.2, and about 1:2.0:3.0 equivalents.

17. The process according to claim 1 wherein the reducing agent is selected from LiAlH₄, NaBH₄, BH₃, and dihydrogen (H₂).

18. The process according to claim 1 wherein compound of formula 5′ produced in Step 4 is in the presence of toluene, heptane, or a mixture of toluene and heptane.

19. The process according to claim 1 wherein the chiral purity of Compound 5′ is >99.5% diastereomeric excess and >99.5% enantioselectivity excess

20. The process according to claim 1 that requires no chromatographic purification.

21. The process according to claim 1 wherein a compound of formula 5′ is made in yields of at least 50%.
